(19) United States
(12) Patent Application Publication
(10) Pub. No.: US 2021/0233651 A1
(43) Pub. Date: Jul. 29, 2021

(54) STORAGE MEDIUM, MEDICAL INFORMATION PROCESSING DEVICE, AND MEDICAL INFORMATION PROCESSING METHOD

(71) Applicant: KONICA MINOLTA, INC., Tokyo (JP)

(72) Inventor: Hiroyuki KUBOTA, Tokyo (JP)

(73) Assignee: KONICA MINOLTA, INC., Tokyo (JP)

(21) Appl. No.: 17/155,213

(22) Filed: Jan. 22, 2021

(30) Foreign Application Priority Data
Jan. 28, 2020 (JP) 2020-011303

(51) Int. Cl. G16H 40/20; G16H 70/20; G16H 30/20; G06F 40/42

(52) U.S. Cl. CPC G16H 40/20; G16H 70/20; A61B 6/52; G06F 40/42; G16H 30/20

(57) ABSTRACT

Provided is a non-transitory storage medium that stores a computer readable program for a computer configured to output and/or input medical information, the program causing the computer to perform: reading the medical information; and determining whether an escape sequence for switching a character encoding is established in the medical information read in the reading.

### CROSS-REFERENCE TO RELATED APPLICATIONS

The entire disclosure of Japanese Patent Application No. 2020-011303 filed on Jan. 28, 2020 is incorporated herein by reference in its entirety.

### BACKGROUND

### Technological Field

The present invention relates to a storage medium, a medical information processing device, and a medical information processing method.

### Description of the Related Art

Generation of medical information in imaging apparatuses, report systems, ordering systems, etc. and exchange of medical information between devices are carried out in conformity with a medical data standard such as Digital Imaging and Communications in Medicine (DICOM), and Health Level Seven International (HL7).

Such medical data standards support character encodings suitable for countries and regions where the devices are used. For example, ASCII is used in the U.S., Latin 1 in France, Europe, Latin 4 in Sweden, also Europe, and JIS for half-width kana, JIS for commonly-used kanji, etc. in Japan.

A character encoding declaration by a device that transmits medical information enables a device that receives the information (destination) to process the encoding in the received information so that the information is output correctly.

However, ASCII does not require the character encoding declaration exceptionally among many encodings, because of its development history. As a result, in some cases, the encoding declaration is omitted by mistake in transmission of medical information that uses an encoding other than ASCII. When a device receives such medical information (destination), the device outputs the medical information in ASCII, the user cannot recognize the output medical information.

In order to deal with such a problem, identification information of a client that receives medical information and a character type(s) that can be recognized by the client are registered beforehand in a server that transmits the medical information, and when the server transmits the medical information, the server refers to the registered identification information and declares the encoding suitable for the client that receives the medical information (for example, see JP2006-004192A).

### SUMMARY

However, the technique disclosed in JP2006-004192A cannot deal with the problem in a case where the server declares an encoding inconsistent with the encoding actually used for the medical information.

Also, in a case where the destination device to be registered beforehand has no specifications published or no maintenance provided, the encoding suitable for the destination device cannot be confirmed, and pre-registration may be difficult.

Further, in a case where medical information is exchanged via a medium, a client(s) and character types cannot be registered beforehand, and an appropriate encoding declaration is impossible.

The present invention has been conceived in view of such problems, and has an object of enabling interpretation of medical information in cases where an encoding declaration by a device that transmits medical information is incorrect or where an encoding for a device that receives information is not registered beforehand.

To achieve at least one of the abovementioned objects, according to an aspect of the present invention, a non-transitory storage medium reflecting one aspect of the present invention stores a computer readable program for a computer configured to output and/or input medical information, the program causing the computer to perform:

reading the medical information; and

determining whether an escape sequence for switching a character encoding is established in the medical information read in the reading.

To achieve at least one of the abovementioned objects, according to another aspect of the present invention, a medical information processing device reflecting one aspect of the present invention includes:

a reader that reads medical information; and

a determination unit that determines whether an escape sequence for switching a character encoding is established in the medical information read by the reader.

To achieve at least one of the abovementioned objects, according to another aspect of the present invention, a medical information processing method reflecting one aspect of the present invention includes:

reading medical information; and

determining whether an escape sequence for switching a character encoding is established in the medical information read by the reading.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram showing a medical information processing system in an embodiment of the present invention;

FIG. 2 is a block diagram showing a medical information processing system in a variation of the present invention;

FIG. 3 is a block diagram showing a medical information processing system in another variation of the present invention;

FIG. 4 is a block diagram showing a medical information processing device of the medical information processing system in FIG. 1, 2, or 3;

FIG. 5 is a flowchart showing steps of character interpretation processing executed by the medical information processing device in FIG. 4; and

FIG. 6 is a list of escape sequence types.

### DETAILED DESCRIPTION OF THE EMBODIMENTS

Hereinafter, an embodiment of the present invention is described with reference to the drawings. However, the scope of the present invention is not limited to the embodiments or illustrated examples.

### [1. Medical Information Processing System]

First, a schematic configuration of a medical information processing system 100 in this embodiment is described.

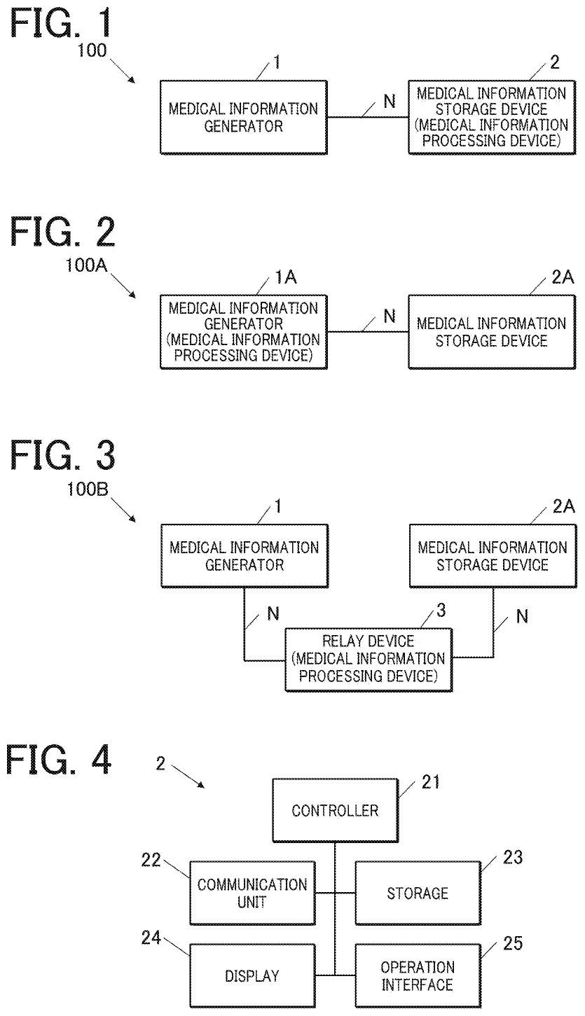

FIG. 1 is a block diagram showing the medical information processing system 100.

The medical information processing system 100 includes a medical information generator 1 and a medical information storage 2.

The medical information generator 1 and the medical information storage 2 can communicate with each other via a communication network N.

The communication network N may be wired or wireless.

The medical information generator 1 and the medical information storage 2 may exchange information using a recording medium (a USB memory, DVD-ROM, etc.) without the communication network N.

The medical information generator 1 in this embodiment is, for example, an imaging apparatus that generates medical images, a report system for creating reports, or an ordering system for entering order information. The medical information generator 1 may generate digital content for medical use.

The “digital content” is text or audiovisual representations of images, figures, audio, video, in the form of digital data expressed by combinations of 0 and 1 in some data format.

For example, audio stored in a storage medium (CD-ROM, etc.) is a kind of the digital content.

The digital content includes computer programs that display/play such content or change it according to the user operation, such as video games.

The digital content may be directly stored or processed in computers, and is easily modified, copied, and transmitted. Moreover, it is less likely to deteriorate due to the state of the recording medium or transmission medium.

The digital content handled by the medical information processing system 100 in this embodiment is at least any of patient information in HL7, order information in HL7, DICOM images (medical images), and DICOM structured documents (reports, etc.).

The digital content includes medical information.

“Medical information,” which is information related to medical practice, specifically includes image information, report information, patient information, order information, clinical test result information, insurance claim information, clinical records, and radiation dose information.

The medical information storage 2 in this embodiment is, for example, a server, or a terminal device.

The medical information storage 2 may store digital content generated by the medical information generator 1.

The medical information storage 2 in this embodiment also functions as a medical information processing device.

The medical information storage 2 is described in detail later.

In FIG. 1, the medical information storage 2 (a computer that can execute input (reception) of medical information) also functions as the medical information processing device, but as shown in FIG. 2, the medical information generator 1A (a computer that can execute output (transmission) of medical information) may also function as the medical information processing device. Alternatively, as shown in FIG. 3, the medical information generator 1 and the medical information storage 2A may be separately provided, and a relay device (a computer that can execute input and output (transfer) of medical information) between the medical information generator 1 and the medical information storage 2A may be provided for communication between the medical information generator 1 and the medical information storage 2A so as to function as the medical information processing device.

### [2. Medical Information Processing Device]

Next, the medical information storage 2 of the medical information processing system 100, as an example of the medical information processing device, is described in detail.

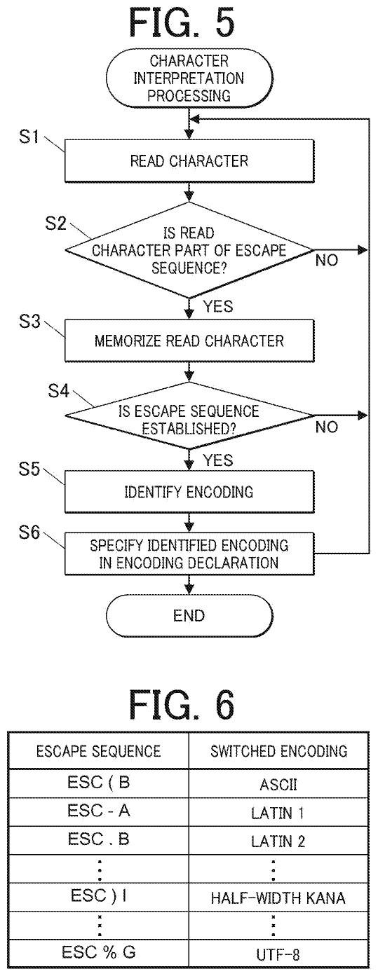

FIG. 4 is a block diagram of the medical information processing device, and FIG. 5 is a flowchart showing steps of character interpretation processing executed by the medical information processing device.

As described above, the medical information processing device may be the medical information generator 1A or the relay device 3.

### (Configuration of Medical Information Processing Device)

The medical information storage 2 includes a controller 21, a communication unit 22, and a memory 23.

The medical information storage 2 in this embodiment further includes a display 24 and an operation interface 25.

The controller 21 includes, for example, a central processing unit (CPU) and a random access memory (RAM).

The CPU of the controller 21 reads out various programs (including a program for executing the character interpretation processing described later) stored in the memory 23, deploys them in the RAM, and executes various kinds of processing according to the deployed programs to centrally control the operation of the components of the medical information storage 2.

The communication unit 22, which includes, for example, a wired communication module and a wireless communication module, may send and receive various signals and various kinds of data (including medical information) to and from other devices (the medical information generator 1, the medical information storage 2, etc.) connected via the communication network N (LAN, WAN, Internet, etc.) wirelessly or by wire.

The medical information storage 2 may include a reading/writing unit that can read and write information on a recording medium instead of the communication unit 22.

The memory 23 includes, for example, a non-volatile semiconductor memory, and a hard disk.

The memory 23 stores programs for execution of the processing by the controller 21, parameters necessary for execution of the programs, etc.

The memory 23 in this embodiment may store image data of radiographs.

The medical information storage 2 includes an image data memory provided independently from the memory 23.

The display 24, which is composed of a monitor including a liquid crystal display (LCD) and a cathode ray tube, displays various images, various kinds of information, etc. according to commands of display signals input from the controller 21.

The operation interface 25 includes a keyboard with cursor keys, number keys, and various function keys, a pointing device such as a mouse, and a touch panel superimposed on the surface of the display 24, and is operable by the user.

The operation interface 25 outputs control signals based on the operation by the user to the controller 21.

In the medical information processing system 100, 100A, or 100B including a terminal device (for example, a tablet terminal) with a display, an operation interface, etc. beside the medical information processing device, the medical information storage 2 may not include the display 24 or the operation interface 25.

### (Operation of Medical Information Processing Device)

The controller 21 of the medical information storage 2 configured as described above has a function of executing the character interpretation processing shown in FIG. 5, triggered by acquisition of medical information from other devices (the medical information generator 1, etc.), input of a predetermined operation on the operation interface 25, etc.

In the character interpretation processing, the controller 21 first executes a reading step (Step S1).

In the reading step, the controller 21 reads medical information acquired from other devices.

In the reading step, the controller 21 in this embodiment reads part of medical information. More specifically, a character in a character string included in medical information.

The controller 21 functions as a reader by execution of the reading step (Step 1).

The execution of the reading step corresponds to “reading” in a medical information processing method.

After reading the medical information, the controller 21 in this embodiment executes a judgment step (Step S2).

In the judgment step, the controller 21 determines whether the read character is part of an escape sequence (ESC).

The “escape sequence” represents a character string that commands control of character output.

More specifically, the escape sequence is not a series of literal characters to be output, but is a special character string that commands change of character encodings, change of character colors, cursor motion, character deletion, etc. at the time of output of characters (to be displayed on the display 24, for example).

The “encoding” is a rule and standard for adding a character code to characters.

The character code includes, for example, ASCII (alphabets/numbers/symbols), Latin 1 (alphabets/western European characters/numbers/symbols), Latin 4 (alphabets/northern European characters/numbers/symbols), JIS for half-width kana (alphabets/symbols/symbols), JIS half-width (alphabets/half-width kanas/numbers/symbols), and JIS for commonly-used kanji (first and second level kanjis).

If the controller 21 determines that the read character is not part of an escape sequence (is a normal character) at this judgment step (Step S2: NO), the controller 21 executes the reading step (Step S1) again.

That is, the controller 21 repeats the reading step until it determines that the read character is part of an escape sequence.

At the second and subsequent reading steps, part (one character) of the medical information that was not read in the previous reading step(s) is read.

Specifically, a character lined next to the character read at the previous reading step is read.

A character to be read may be lined apart from the character read at the previous reading step by one or more characters.

On contrary, if the controller 21 determines that the read character is part of the escape sequence at the judgment step (Step S2: YES), the controller 21 stores the read character in the memory 23 or the like (Step S3).

After the read character is stored, the controller 21 executes a determination step (Step S4).

At the determination step, the controller 21 determines whether the escape sequence is established in the medical information read at the reading step in this embodiment.

More specifically, the controller 21 determines whether a character next to the character read at this step (ESC) indicates some kind of the encoding (whether it forms a character string exemplified in FIG. 6, combined with the read character).

The controller 21 functions as a determination unit by execution of the determination step (Step S2).

The execution of the determination step corresponds to “determining” in the medical information processing method.

If the controller 21 determines that the escape sequence is not established at the determination step (Step S4: NO), the controller 21 executes the reading step (Step S1) again.

On contrary, if the controller 21 determines that the escape sequence is established in the medical information (Step S4: YES), the controller 21 in this embodiment executes an identification step (Step S5).

The controller 21 identifies the encoding after being switched at the identification step.

The controller 21 functions as an identifier by execution of the identification step (Step S5).

The execution of the identification step corresponds to “identifying” in the medical information processing method.

After the encoding is identifies, the controller 21 in this embodiment executes a specifying step (Step S6).

The controller 21 specifies the identified encoding in an encoding declaration of the medical information.

The controller 21 functions as a specifying unit by execution of the specifying step (Step S6).

The execution of the specifying step corresponds to “specifying” in the medical information processing method.

The controller 21 executes the character interpretation processing as described hereinbefore until the last character in the acquired medical information (or the last character in a predetermined range).

### [3. Effects]

The medical information processing device as described hereinbefore (the medical information storage 2, the medical information generator 1A, the relay device 3) does not refer to the encoding declaration by the device that transmits the medical information, but finds an escape sequence in the medical information and outputs the medical information based on the escape sequence.

This makes it possible to interpret medical information even in cases where an encoding declaration by a device that transmits medical information is incorrect or where an encoding for a device that receives information is not registered beforehand, in the medical information processing device in this embodiment.

The medical information processing device in this embodiment executes the identification step for identifying the encoding after being switched and the specifying step for specifying the identified encoding in an encoding declaration of the medical information.

This makes it possible for the device that receives medical information to output the medical information correctly, in the medical information processing device in this embodiment.

### [4. Misc.]

It is needless to mention that the present invention is not limited to the above embodiment and can be appropriately modified without departing from the scope of the present invention.

For example, in the above embodiment, the present invention is described as the medical information processing device of the medical information processing system 100, 100A, or 100B. However, the present invention may be applied to various devices and systems.

For example, the present invention may be applied to an imaging apparatus, an image server, an electronic clinical record, an RIS, a clinical examination department system, an image interpretation report system, a medical expense management computer, a gateway device, etc.

The medical information processing device may not necessarily execute the character interpretation processing all over again every time the medical information is transmitted and received. For example, in the case where the medical information is exchanged multiple times (for example, 100 times) and the tendency of usage of the encoding is detected in a medical facility, the steps beside the specifying step (Step S6) may be at least omitted in the character interpretation processing.

Further, in the above description, a hard disk, a non-volatile semiconductor memory, etc. are used as a computer readable medium of the programs of the present invention, but the embodiments are not limited to this example. A portable recording medium such as a CD-ROM can be applied as other computer readable media.

A carrier wave is also applied as a medium providing the program data according to the present invention via a communication line.

What is claimed is:

1. A non-transitory storage medium that stores a computer readable program for a computer configured to output and/or input medical information, the program causing the computer to perform:
reading the medical information; and
determining whether an escape sequence for switching a character encoding is established in the medical information read in the reading.

2. The non-transitory storage medium according to claim 1, wherein the program further causes the computer to perform:
identifying an encoding designated in the escape sequence in response to determination that the escape sequence is established in the medical information in the determining.

3. The non-transitory storage medium according to claim 2, wherein the program further causes the computer to perform:
specifying the identified encoding in an encoding declaration after the identifying.

4. The non-transitory storage medium according to claim 1,
wherein the reading includes reading part of the medical information,
wherein the determining includes determining whether the escape sequence is established based on the part of the medical information read in the reading,
wherein the program causes the computer to repeat the reading in response to determination, in the determining, that the escape sequence is not established,
wherein the repeated reading includes reading part of a rest of the medical information that is not read in the reading previously performed.

5. The non-transitory storage medium according to claim 4, wherein the program further causes the computer to perform:
identifying the encoding designated in the escape sequence in response to determination, in the determining, that the escape sequence is established in the part of the medical information.

6. The non-transitory storage medium according to claim 4,
wherein the medical information is included in at least one of:
a medical image generated by an imaging apparatus;
a report created in a report system; and
order information generated by an ordering system.

7. A medical information processing device comprising:
a reader that reads medical information; and
a determination unit that determines whether an escape sequence for switching a character encoding is established in the medical information read by the reader.

8. The medical information processing device according to claim 7, further comprising:
an identifier that identifies the encoding designated in the escape sequence in response to determination by the determination unit that the escape sequence is established.

9. The medical information processing device according to claim 8, further comprising:
a specifying unit that specifies, after the identifier identifies the encoding, the identified encoding in an encoding declaration.

10. A medical information processing method further comprising:
reading medical information; and
determining whether an escape sequence for switching a character encoding is established in the medical information read by the reading.

11. The medical information processing method according to claim 10, further comprising:
identifying the encoding designated in the escape sequence in response to determination, in the determining, that the escape sequence is established.

12. The medical information processing method according to claim 11, further comprising:
specifying, after the encoding is identified in the identifying, the identified encoding in an encoding declaration.
